(19) United States
(12) Patent Application Publication
(10) Pub. No.: US 2009/0093821 A1
(43) Pub. Date: Apr. 9, 2009

(54) Slide Guide Apparatus & Method

(75) Inventor: Gregory K. Edmundson, Rough & Ready, CA (US)

Correspondence Address: CYTYC CORPORATION;Darry Pattinson, Sr. IP Paralegal, 250 CAMPUS DRIVE, MARLBOROUGH, MA, 01752, US

(73) Assignee: Cytyc Corporation, Marlborough, MA (US)

(21) Appl. No.: 11/866,483

(22) Filed: Oct. 3, 2007

(51) Int. Cl. A61B 19/00

(52) U.S. Cl. 606/108; 606/129

(57) ABSTRACT

Methods of maintaining a consistent pathway through an already formed incision and for facilitating placement and removal of medical devices into and/or out of a surgical treatment site or cavity are disclosed. A method for removing and placing medical devices includes inserting a first medical device into an already formed incision, inserting a slide guide into the cavity through the already formed incision in surrounding relation to the first medical device to facilitate removal of the first medical device, and inserting a second medical device into the cavity using the slide guide as the pathway to guide and partially surround a circumference of the second medical device to guide the second medical into the cavity. A slide guide includes an elongated insertion member having a substantially U-shaped cross-section sized to receive and partially surround a circumference of a tubular insertion member of a medical device.

### TECHNICAL FIELD

This technology related generally to the methods and devices which facilitate the insertion and removal of medical devices from a surgical site.

### BACKGROUND

Body tissues subject to proliferative tissue disorders, such as malignant tumors, are often treated by surgical resection of the tumor as possible. Unfortunately, the infiltration of the tumor cells into the normal tissues surrounding the tumor may limit the therapeutic value of surgical device resection because the infiltration can be difficult or impossible to treat surgically. Radiation therapy may be used to supplement surgical resection by targeting the residual tumor after resection, with the goal of reducing its size or stabilizing it. Radiation therapy may be administered through one of several methods, or a combination of methods, such as interstitial or intercavity brachytherapy. Brachytherapy may also be administered using radioactive sources, such as x-ray sources, for example.

Brachytherapy is the delivery of a course of radiation therapy to a body site by placing the radiation source into the body site or surgical treatment area, such as a cavity left after surgical reaction of a tumor. Usually, brachytherapy is performed by inserting an implantable brachytherapy device (e.g., catheter or applicator) into the surgical treatment site and later a radiation source is inserted through the implantable device and into the surgical treatment site.

A typical brachytherapy device applicator catheter comprises a tubular member having a distal portion which is adapted to be inserted into the patient's body, and a proximal portion which extends outside of the patient. A balloon may be provided on the distal portion of the tubular member which, when placed at the treatment site and inflated, causes the surrounding tissue to substantially conform to the surface of the balloon. In use, the distal portion of the tubular member of the applicator catheter having die balloon thereon is placed at, or near, the treatment site, e.g. the resected tumor cavity. The balloon is inflated, and a radiation source is placed through the tubular member to the location within the balloon. The radiation source within the balloon creates an isodose profile and treats the tissues surrounding the resected tumor cavity.

In some situations, before inserting a balloon-based implantable brachytherapy device, it may be desirable to use another device to measure or evaluate the size, shape or other dimensions of the surgical cavity to be treated. This device, sometimes called a cavity evaluation device, may also have a tubular applicator or catheter to provide access into the surgical treatment site. In the situation where a cavity evaluation device is used in conjunction with an implantable brachytherapy treatment device, there remains a need to provide consistent placement for both devices. Consistent placement for both medical devices within the surgical cavity may be accomplished by providing a consistent pathway through an incision. Further, there remains a need to facilitate both the removing and placing of multiple devices at the surgical treatment site via an incision.

Disclosed herein are devices and methods for removing and placing medical devices and for maintaining a consistent pathway to a surgical treatment site.

### SUMMARY

Methods and devices which facilitate the insertion and removal of medical devices from a surgical cavity are disclosed herein. In one embodiment, a method for removing and placing medical devices comprises: i) inserting a first medical device into a cavity through an already formed incision, wherein the first medical device has a tubular insertion member; ii) inserting a slide guide into the cavity through the already formed incision in adjacent and surrounding relation to the first medical device, wherein the slide guide has an elongated insertion member having a substantially U-shaped cross-section and is positioned to partially surround an outer circumference of the tubular insertion member of the first medical device to maintain a pathway through the already formed incision to facilitate removal of the first medical device; and iii) inserting a second medical device into the cavity through the already formed incision using the substantially U-shaped cross-section of the slide guide as the pathway to guide and partially surround a circumference of a tubular insertion member of the second medical device to guide the second medical device through the already formed incision and into the cavity.

In another embodiment, a method for maintaining a consistent pathway through an already formed incision, computer: i) inserting a first medical device into a cavity through an already formed incision, wherein the first medical device has a tubular insertion member; ii) inserting a slide guide into the cavity through the already formed incision in adjacent and surrounding relation to the first medical device, wherein the slide guide has an elongated insertion member having a substantially U-shaped cross-section and is positioned to partially surround an outer circumference of the tubular insertion member of the first medical device to maintain a consistent pathway through the already formed incision; and iii) removing the first medical device.

In yet another embodiment, a slide guide apparatus to facilitate placement and removal of medical device in a cavity through an already formed incision is disclosed. The slide guide includes an elongated insertion member having a substantially U-shaped cross-section sized to receive and partially surround a circumference of a tubular insertion member of a medical device.

The methods and slide guides disclosed herein facilitate the insertion and/or removal of medical devices into and/or out of surgical incisions and cavities. When performing brachytherapy, treatment devices may include an elongated tubular portion (i.e., catheter). The slide guides disclosed herein will provide a pathway for slideable insertion and removal of brachytherapy catheters into and/or out of surgical incisions and cavities. Additionally, when performing brachytherapy it may be desirable to use more than one brachytherapy treatment related device, such as a cavity evaluation device to determine the size and shape of the surgical cavity to be treated. When using more than one brachytherapy treatment related device it would be advantageous to maintain a consistent pathway through an incision to ensure consistent placement of multiple brachytherapy treatment related devices in the same or similar positions within the cavity. The methods and devices disclosed herein may also be used to maintain a consistent pathway through an already formed incision to a surgical treatment site or cavity.

This summary is provided to introduce a selection of concepts in a simplified form that are further described below in the Detailed Description. This summary of is not intended to identify key features or essential features of the claimed subject matter, nor is it intended to be used to limit the scope of the claimed subject matter.

### BRIEF DESCRIPTION OF THE DRAWINGS

FIG. 1 illustrates a perspective view of an exemplary slide guide;

FIG. 2A illustrates a cross-sectional view of a U-shaped section of an exemplary slide guide;

FIG. 2B illustrates a cross-sectional view of a C-shaped section of an exemplary slide guide;

FIG. 3A illustrates a top view of an exemplary slide guide;

FIG. 3B illustrates a top view of an exemplary slide guide;

FIG. 4 illustrates a perspective view of an exemplary method of using a slide guide for insertion/removal of a brachytherapy treatment device into a surgical cavity;

FIG. 5 is a flow diagram illustrating an exemplary method for removing and placing medical devices; and

FIG. 6 is a flow diagram illustrating an exemplary method for maintaining a consistent pathway through an already formed incision to a surgical treatment site.

### DETAILED DESCRIPTION

Slide guides and methods for using the slide guide to remove and place medical devices through an incision, as well as methods for maintaining consistent medical device placement, are disclosed herein. The slide guides disclosed herein provide a guide or pathway for slideable insertion and removal of brachytherapy catheters into and/or out of surgical incisions and cavities. These slide guides also provide a consistent pathway through an incision to provide more consistent placement of multiple brachytherapy catheters within a surgical treatment site or cavity. Referring now to the drawing figures, like numerals indicate like features throughout the drawing figures shown and described herein.

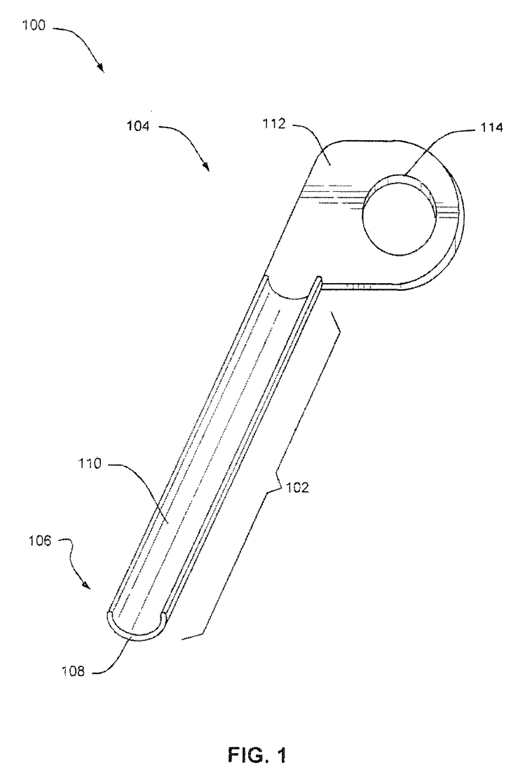

With reference to FIG. 1, a slide guide 100 may comprise an elongated insertion member 102 having a proximal end 104 and a distal end 106. The distal end 106 is adapted to be inserted through an already formed incision into a surgical treatment site or cavity in a patient's body. The distal end 106 may be rounded or shaped to prevent puncturing or rupturing of a catheter of a brachytherapy device during use. In alternative embodiments, the distal end 106 may be formed to have a more pointed or even sharpened end to facilitate entry through an incision. The proximal end 104 of the slide guide 100 is adapted to extend outside of the incision and to remain outside the patient's body during treatment. Additionally, the elongated insertion member 102 may also comprise a guide member 112 (described in more detail below) to help guide or control insertion of the slide guide 100 through an already formed incision and into a surgical treatment site.

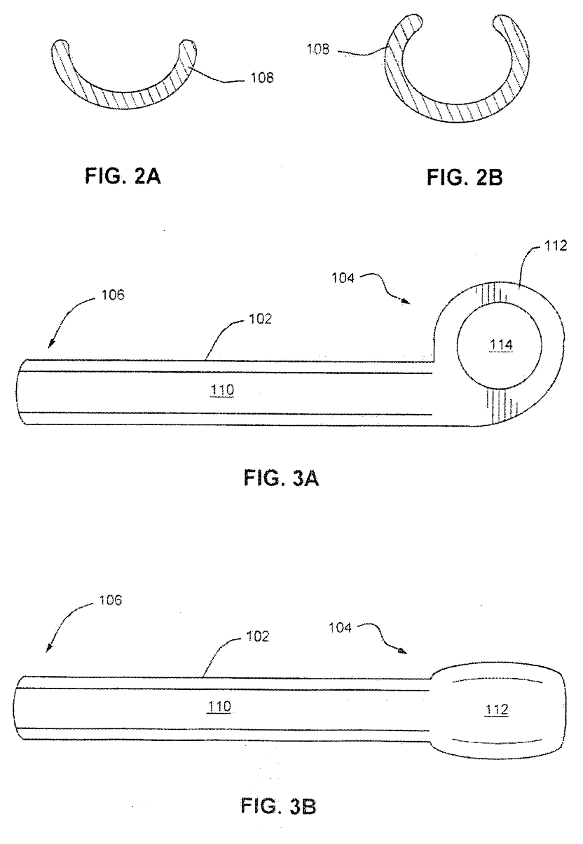

The elongated insertion member 102 may have a substantially U-shaped cross-section 108, as shown in FIGS. 1 & 2A. The substantially U-shaped cross-section 108 of the elongated insertion member provides a substantially smooth channel or trough 110 to slideably receive and partially surround a circumference of a tubular insertion member or catheter of medical devices. The U-shaped cross-section 108 provides a smooth and uniform pathway 110 or groove 110 to facilitate slideable insertion and removal of those medical devices through an incision. In alternative embodiments, the cross-section of the elongated insertion member 102 may have a number of different shapes and may, for example, be substantially C-shaped (see FIG. 2B), horseshoe shaped, V-shaped, have flared or flanged edges, or may even have very minimal curvature.

The elongated insertion member 102 may be formed of any appropriate biocompatible material(s), such as polymers, elastomeric and/or metallic materials, for example. Additionally, the elongated insertion member 110 may be formed to be flexible, rigid, or semi-rigid. The walls 108 of the elongated insertion member 102 may or may not have a uniform thickness and/or circumference (i.e., perimeter) for its entire length. As shown in FIG. 1, the elongated insertion member 102 may be formed to be substantially straight or linear, but in other embodiments may also be formed with curved or shaped portions. The curved or shaped portions of the elongated insertion member 102 may be positioned on either or both of the distal 106 and/or proximal ends 114 and may further be designed to match particular physical characteristics of some surgical treatment sites to be more comfortable for patients. It should be understood that the elongated insertion member 102 may have any number of sizes and shapes which may be modified depending upon the final desired application of tile slide guide 100.

In some implementations, the elongated insertion member 102 may be formed of a flexible material to ensure it is flexible and soft enough to conform to surrounding tissues and bends easily when force is applied, such as by movement of the patient's body or articulation by a physician, making the elongated insertion member 102 more comfortable. Having a flexible elongated insertion member 102 may also allow the elongated insertion member 102 to be articulated or bent to compensate for protrusions or other features, on medical devices and/or in a patient's body. In some implementations, the distal portion of a brachytherapy device may comprise an expandable balloon and thus, it may be beneficial to slightly bend or articulate the elongated insertion member 102 to help ease the balloon portion of the brachytherapy device through the incision.

The elongated insertion member 102 may further comprise a malleable element (not shown) adapted to confer a shape upon at least a portion of its length. The malleable element may also extend the entire length of the slide glide 102 and may be used to bend or curve the proximal end 104 of the slide guide 100 by folding it to lay flat again a patient's skin during treatment. In other embodiments, the walls 108 of the elongated insertion member 102 may be substantially impermeable to fluids and/or may be coated or lubricated to ease insertion of medical devices through the U-shaped channel or pathway 110. In some embodiments, it may even be desirable to use one or more slide guides 100 simultaneously. For example, two slide guides 100 may be positioned adjacent one another to form an elongated hollow cylindrical tube (e.g., with each U-shaped channel 110 forming a half of the hollow cylindrical tube) through an incision.

In some situations, the walls 108 of the elongated insertion member 102 may even be formed of a radiation transparent material to allow radiation to pass through the walls thereof to treat issues surrounding the surgical treatment without interference or interruption by the elongated insertion member 102. Alternatively, the elongated insertion member 102 may compromise radiopaque materials or markers to help locate the exact position of the elongated insertion member 102 using X-ray or other electronic imaging techniques. Additionally, the elongated insertion member 102 may compromise measurement or indicator markings disposed along its length to provide a way to measure or maintain consistent depth of the elongated insertion member 102.

The elongated insertion member 102 may have a length longer than, shorter than, or substantially similar to, that of the medical devices with which it will be used. In some implementations, the elongated insertion member 102 may be used to help insert and remove brachytherapy devices. In this implementation, the elongated insertion member 102 may have a length similar to the length of the tubular insertion member (e.g., catheter or applicator) of the brachytherapy device.

The slide guide 100 may further comprise a guide member 112, as shown in FIG. 1. The guide member 112 may be positioned on the proximal end 104 and may be used to provide a mechanism for grasping the slide guide 100 and for guiding placement of the slide guide 100 through an incision and into a surgical treatment site. A physician may use the guide member 112 to direct and control precise placement of the slide guide 100 through an incision and to a surgical treatment site.

In some implementations, the guide member 112 may comprise an opening or finger loop 114 sized for receiving a finger, as shown in FIG. 3A. The finger loop 114 may comprise a thin wire or elastomeric loop sized for receiving one or more fingers. In some embodiments, the guide member 112 may comprise a plurality of loops or one large loop sized for receiving multiple fingers and/or a hand to guide placement of the slide guide 100. In other implementations, the guide member 112 may comprise a protrusion or paddle portion which may be sized for receiving a thumb, as shown in FIG. 3B.

Guide member 112 may be attached to elongated insertion member 102 and/or may be formed to be integral with the elongated insertion member 102 and may or may not be comprised of the same materials. Additionally, the guide member 112 may be flexibly or releasably attached to the elongated insertion member 102 so that it may be folded during treatment and/or during packaging to make the slide guide 100 more compact. During treatment, the guide member 112 may be laid flat to be positioned parallel to a patient's skin to provide more space for a physician to work and/or help the slide guide 100 maintain a constant position (e.g., by not slipping further into or out of the incision). Guide member 112, may be formed in a number of different configurations and may have any number of sizes and shapes, as will be apparent to those of ordinary skill in the art after having become familiar with the teachings herein.

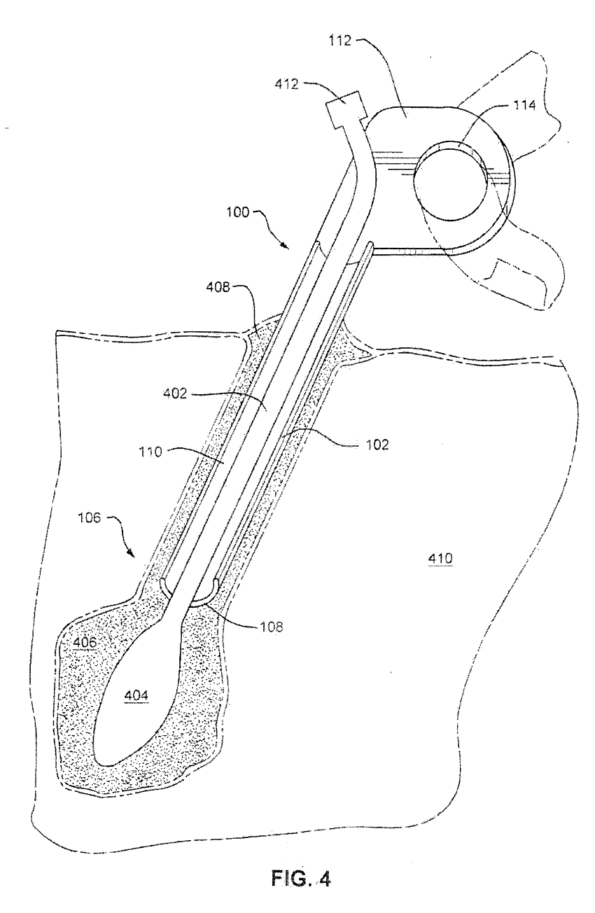

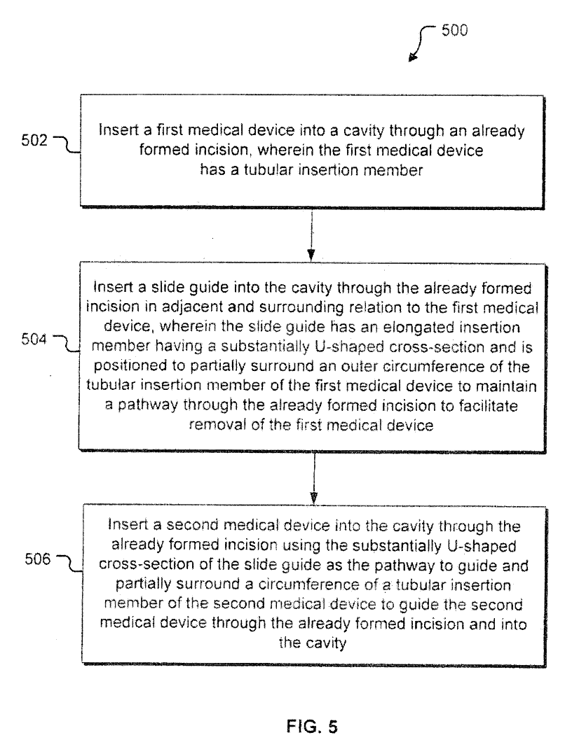

With reference now to FIGS. 4, 5 & 6, method(s) 500/600 for facilitating the removal and insertion of medical devices through an incision and for maintaining a consistent pathway through an incision are shown and described. The slide guide 100 may be used to help ease the insertion and removal of medical devices through an already formed incision and into a surgical treatment site or cavity in a patient. The medical devices to be inserted and removed may be any type of medical device comprising an elongated tubular insertion member, such as a brachytherapy device. The slide guide 100 may also be used to maintain a continuous and consistent pathway through an incision to maintain consistent placement of multiple medical devices within a surgical cavity.

As shown in FIG. 4, a medical device to be inserted and removed may comprise a brachytherapy device 402 such as those used for performing brachytherapy on a cavity 406 left after resection of a tumor. The slide guide 100 provides a smooth continuous channel or pathway (shown generally as 110) through an incision 408 leading to the cavity or surgical treatment site 406 within a patient (shown generally as tissue 410). In some cases, the incision 408 may have already been formed, such as during surgical rescission of the tumor.

FIG. 4 generally illustrates the placement of a medical device (e.g., either a first or second medical device) and, for simplicity of illustration herein, may be viewed generally as either a cavity evaluate device or a brachytherapy treatment device. During typical brachytherapy treatment procedures, a cavity evaluate device (e.g., a first medical device) will be inserted before insertion of the brachytherapy device (e.g., a second medical device).

During treatment, a method 500/600 of using the slide guide 100 may begin by inserting 504/604 a first medical device 402 into the cavity 406 through an already formed incision 408. A physician may insert the first medical device 402 by pushing the first medical device 402 into the incision 408. The first medical device 402 has a tubular insertion member (shown generally as 402) and may also comprise a (deflated) balloon 404 on its distal end 106. After inserting 504/604 a first medical device 402 into the cavity 406, a slide guide 100 may then be inserted 506/606 into the cavity 406 through the already formed incision 408 an adjacent and surrounding relation to the first medical device 402, as shown in FIG. 4.

A physician may insert the slide guide 100 by pushing on the slide guide 100 to slide it into the incision 408 in surrounding relation to the tubular insertion member of the first medical device 402. In some situations, it may be desirable for the physician to articulate, rotate, and/or wiggle the slide guide 100 while pushing down on the slide guide 100 during insertion 504/604. The slide guide 100 has an elongated insertion member 102 having a substantially U-shaped cross-section 108 positioned to partially surround an outer circumference of the tubular insertion member 402 of the first medical device 402 to maintain a consistent pathway (shown generally as incision 408) through the already formed incision 408 to facilitate removal of the first medical device 402. The slide guide 100 may have an elongated insertion member 102 designed to substantially conform to the U-shaped cross-section 108 of the tubular insertion member of the first medical device 402.

After inserting 504/604 the slide guide 100 through the incision 408, a second medical device (not shown in FIG. 4) may then be inserted 506 through the incision 408. However, in some implementations, it may be desirable to remove 606 the first medical device 402 before inserting a second medical device. Alternatively, there may also be room in the incision 408 to insert more than one medical device at a time. When inserting a second medical device (not shown in FIG. 4) through the incision 408 and into the cavity 406 the slide guide 100 may again be used or pathway 110, wherein the substantially U-shaped 108 of the elongated insertion member 102 partially surrounds a circumference of a tubular insertion member of the second medical device. A physician may insert the second medical device by pushing it into the incision in adjacent relation to the slide guide 100.

In some implementations, it may be desirable to remove 606 the first medical device 402 before inserting 506 a second medical device. A physician may remove 606 the first medical device 402 by pulling on the medical device 402 to slide it in relation to the U-shaped cross-section 108 or channel 110 (e.g., the slide guide 100) to slideably pull the device 402 out of the incision 408. Upon completion of the medical procedure using the second medical device, the second medical device may also be removed by a physician by pulling on the second medical device to slide the tubular insertion member of the second medical device in relation to the U-shaped section 108 of the slide guide 100 to remove the second medical device from the patient. Upon completion of all medical procedures at the surgical treatment site 406, a physician may remove the slide guide 100 by pulling it out of the incision 408.

While the above described methods of treatment 500/600 disclose inserting 502/602 a first medical device 402 before inserting a slide guide 100, in some situations it may be desirable to insert the slide guide 100 before inserting the first medical device 402. In this situation,the slide guide 100 may be more rigid and thus, may be easier to insert through an incision 408 initially.

When inserting and/or removing the medical devices 402 and/or the slide guide 100 a physician may be pushing or pulling on the medical devices 402 and/or the slide guide 100 simultaneously. Additionally, the physician may also want to slightly articulate or rotate one or more of the medical devices 402 and/or slide guide 100 while pushing or pulling. To provide a physician with better control over guiding and placing the slide guide 100, a guide member 112 is disposed on the proximal end 104 thereof. As described above, the guide member 112 may provide a protrusion or other easily grasped area, such as a finger loop 114 or paddle, to provide means for guiding the slide guide 100 accurately through the incision 408. Additionally, the guide member 112 may be bent or manipulated to lie parallel to or flat against (not shown) the surface of a patient's skin during treatment, providing a physician with better access to the proximal end 104 if a medical device 402, such as to port 412, show in FIG. 4.

The use of the slide guide 100 may help to place multiple medical devices 402 in substantially similar positions within the cavity 406. The consistent placement of subsequent medical devices may help to provide more effective brachytherapy treatments for patients by providing physicians and other medical professionals with more accurate dimensions and measurements when calculating an isodose profile, for example.

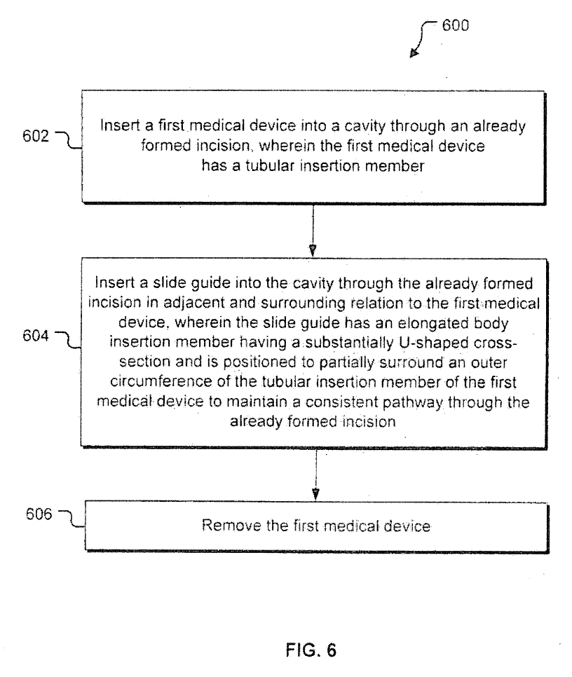

With reference now to FIG. 6, a method 600 for maintaining a consistent pathway through an already formed incision includes inserting 602 a first medical device 402 into a cavity 406 through an already formed incision 408, wherein the first medical device 402 has a tubular insertion member 102. The method 600 continues by inserting 604 a slide guide 100 into the cavity 406 through the already formed incision 408 in adjacent and surrounding relation to the first medical device 402. The substantially U-shaped cross-section 108 of the elongated insertion member 102 is positioned to partially surround an outer circumference of the tubular insertion member 102 of the first medical device 402 to maintain a consistent pathway through the already formed incision. The method 600 continues be removing 606 the first medical device 402 from the incision 408 using the slide guide 100 as a pathway.

When using the slide guide 100 the administer brachytherapy treatment, it may be desirable to: 1) insert a cavity evaluation device (e.g., first medical device); 2) insert a slide guide 100 in adjacent and surrounding relation to the cavity evaluation device; 3) remove the cavity evaluation device by pulling and sliding it in relation to the slide guide 100; 4) insert a brachytherapy device by pushing and sliding it into the incision, using the slide guide 100 as a pathway; 5) administering brachytherapy treatment; 6) upon completion of the brachytherapy treatment, removing the brachytherapy device by pulling and sliding it in relation to the slide guide 100; and 7) removing the slide guide 100 from the patient by pulling it out of the incision. In an alternative embodiment, steps #1 and #2 above may be reversed, and the slide guide 100 may be inserted first and the cavity evaluation device may be inserted second.

In yet another embodiment, methods for removing and placing medical devices and/or maintaining a consistent pathway to a medical treatment site may include forming an incision to access the medical treatment site or cavity. The incision may be formed using a device having a sharpened elongated insertion member, such as a trocar for example. The trocar may be used to form an incision to provide an access pathway to the medical treatment site and may be used in conjunction with the slide guide. The slide guide may be positioned in adjacent and surrounding relation to the sharpened tubular insertion member of the trocar, such that the substantially U-shaped cross-section of the elongated insertion member of the slide guide partially surrounds an outer circumference of the tubular insertion member of the trocar during formation of the incision. The slide guide may then remain in place while the trocar is removed from the incision. The trocar may be removed by sliding it in relation to the U-shaped cross-section of the side guide, to pull the trocar out of the incision.

The slide guide may remain in place after removal of the trocar to maintain a consistent pathway to a medical treatment site through the incision formed by the trocar. Once the trocar is removed, a medical device may then be inserted into the medical treatment site using the substantially U-shaped cross-section of the slide guide as the pathway to guide the medical device into the medical treatment site, thus providing a consistent pathway to the medical treatment site. In some embodiments, this medical device may then be removed, using the slide guide as a pathway, an yet another medical device may be inserted, again using the slide guide as a pathway, thus facilitating the insertion/placement and removal of medical devices into/from the medica treatment site.

Also disclosed herein are kits, such as kits if component parts, for removing and placing medical devices at a medical treatment site. A kit may comprise a torcar, a slide guide, and a brachytherapy treatment device. The trocar may have a sharpened elongated tubular insertion member for forming an incision to access a medical treatment site. The slide guide may have an elongated insertion member having a substantially U-shaped cross section sized to partially surround an outer circumference of the elongated tubular insertion member of the trocar to maintain a consistent pathway through the incision to facilitate removing and placing medical devices at the medical treatment site. The brachytherapy treatment device may have an elongated tubular insertion member sized to be received by and partially surrounded by the substantially U-shaped cross-section of the slide guide, wherein the brachytherapy treatment device has a distal end and a proximal end and an expandable chamber disposed on the distal end of the tubular insertion member.

Disclosed herein are devices and methods for use facilitating insertion and removal of medical devices and for use in maintaining a consistent pathway through an incision. While the devices and methods disclosed herein are particularly useful in providing a pathway through an already formed incision and into a surgical treatment site, such as that of a resected tumor, a person skilled in the art will appreciate that the methods and devices disclosed herein can have a variety of configurations, and they can be adapted for use in a variety of medical procedures. The devices and methods disclosed herein may also be used for non-human medical procedures, such as for veterinary medical procedures, for example.

It should be understood that various changes and modifications to the above-described embodiments will be apparent to those skilled in the art. The examples and illustrations given herein are not meant to be limiting, but rather exemplary of the modifications that can be made without departing from the spirit scope of the described embodiments and without diminishing its attendant advantages.

What is claimed is:

1. A method for removing and placing devices, comprising:
inserting a first medical device into a cavity through an already formed incision, wherein the first medical device has a tubular insertion member;
inserting a slide guide into the cavity through the already formed incision in adjacent and surrounding relation to the first medical device, wherein the slide guide has an elongated insertion member having a substantially U-shaped cross-section and is positioned to partially surround an outer circumference of the tubular insertion member of the first medical device to maintain a pathway through the already formed incision to facilitate removal of the first medical device; and
inserting a second medical device into the cavity through the already formed incision using the substantially U-shaped cross-section of the slide guide as the pathway to guide and partially surround a circumference of a tubular insertion member of the second medical device to guide the second medical device through the already formed incision and into the cavity.

2. A method of claim 1, wherein inserting the second medical device into the cavity using the substantially U-shaped cross-section of the slide guide as the pathway places the second medical device in a substantially similar position within the cavity as the first medical device due to consistent pathway placement through the incision.

3. The method of claim 1, further comprising the step of removing the first medical device after inserting the slide guide and before inserting the second medical device.

4. The method of claim 3, wherein removing the first medical device further comprises pulling on the first medical device to slide the tubular insertion member of the first medical device in relation to the U-shaped cross-section of the slide guide to facilitate removal of the first medical device.

5. The method of claim 1, wherein inserting the first medical device further comprises sliding the tubular insertion member of the first medical device in relation to the U-shaped cross-section of the slide guide to facilitate placement of the first medical device.

6. The method of claim 1, wherein inserting the slide guide into the cavity further comprises disposing the slide guide to substantially conform to the U-shaped cross-section of the tubular insertion member of the first medical device.

7. The method of claim 1, further comprising guiding insertion of the slide guide through the already formed incision using a guide member positioned on a proximal end of the elongated insertion member.

8. The method of claim 1, further comprising removing the slide guide from the incision upon completion of a medical procedure.

9. The method of claim 1 wherein inserting the second medical device comprises inserting a brachytherapy treatment device.

10. The method of claim 1, wherein inserting the first medical device comprises inserting a cavity evaluation device.

11. A method of maintaining a consistent-pathway through an already formed incision comprising:
inserting a first medical device into a cavity through an already formed incision, wherein the first medical device has a tubular insertion member;
inserting a slide guide into the cavity through the already formed incision in adjacent and surrounding relation to the first medical device, wherein the slide guide has an elongated insertion member having a substantially U-shaped cross-section and is positioned to partially surround an outer circumference of the tubular insertion member of the first medical device to maintain a consistent pathway through the already formed incision; and
removing the first medical device.

12. A method of claim 11, further comprising inserting a second medical device into the cavity using the substantially U-shaped cross-section of the slide guide as the pathway to place the second medical device in a substantially similar position within the cavity as the first medical device occupied.

13. The method of claim 12, wherein inserting the second medical device furher comprises sliding the tubular insertion member of the second medical device in relation to the U-shaped cross-section of the slide guide to facilitate placement of the second medical device.

14. The method of claim 11, wherein removing the first medical device further comprises pulling on the first medical device to slide the tubular insertion member of the first medical device in relation to the U-shaped cross-section of the slide guide to facilitate removal of the first medical device.

15. The method of claim 11, wherein inserting the slide guide into the cavity further comprises disposing the slide guide with the U-shaped cross-section of the elongated insertion member substantially conforming to the tubular insertion member of the first medical device.

16. The method of claim 11, further comprising guiding insertion of the slide guide through the already formed incision using a guide member positioned on a proximal end of the elongated insertion member.

17. The method of claim 11, further comprising removing the slide guide from the incision upon completion of a medical procedure.

18. The method of claim 11, wherein inserting the first medical device comprises inserting a brachytherapy treatment related device.

19. A slide guide apparatus for facilitating placement and removal of medical devices in a cavity through an already formed incision comprising: an elongated insertion member having a substantially U-shaped cross-section sized to receive and partially surround a circumference of a tubular insertion member of a medical device.

20. The slide guide apparatus of claim 19, wherein the elongated insertion member having a substantially U-shaped cross-section is substantially rigid.

21. The slide guide apparatus of claim 19, wherein the elongated insertion member having a substantially U-shaped cross-section is substantially flexible.

22. The slide guide apparatus of claim 19, wherein a proximal end of the elongated main body portion further comprises a guide member to more precisely guide placement of the slide guide through the already formed incision.

23. The slide guide apparatus of claim 19, wherein the guide member comprises a loop sized for receiving a finger.

24. A kit for removing aid placing medical devices at a medical treatment site, comprising:
a trocar having a sharpened elongated tubular insertion member for forming an incision to access a medical treatment site:
a slide guide having an elongated insertion member having a substantially U-shaped cross-section sized to partially surround an outer circumference of the elongated tubular insertion member of the trocar to maintain a consistent pathway through the incision to facilitate removing and placing medical devices at the medical treatment site; aid
a brachytherapy treatment device having an elongated tubular insertion member sized to be received by and partially surrounded by the substantially U-shaped cross-section of the slide guide, wherein the brachytherapy treatment device has a distal end and a proximal end and an expandable chamber disposed on the distal end of the tubular insertion member.

25. A method of maintaining a consistent pathway to a medical treatment site, comprising:
inserting a trocar to form an incision to a medical treatment site, the trocar having a slide guide positioned in adjacent and surrounding relation to a sharpened tubular insertion member of the trocar, wherein the slide guide has an elongated insertion member having a substantially U-shaped cross-section to partially surround an outer circumference of the tubular insertion member of the trocar, wherein the slide guide maintains a consistent pathway to a medical treatment site through the incision formed by the trocar;
removing the trocar from the incision; and
inserting a medical device into the medical treatment site using the substantially U-shaped cross-section of the slide guide as the pathway to guide and partially surround a circumference of a tubular insertion member of the medical device to guide the medical device to the medical treatment site.
